(19) United States
(12) Patent Application Publication
(10) Pub. No.: US 2006/0287732 A1
(43) Pub. Date: Dec. 21, 2006

(54) Maxillary sinus bone augmentation with resorbable bone pack

(76) Inventor: Alex Ahmad Pezeshkian, Cypress, CA (US)

Correspondence Address: RONALD M. GOLDMAN;ROTH & GOLDMAN, SUITE 500, 21535 HAWTHORNE BLVD., TORRANCE, CA, 90503, US

(21) Appl. No.: 11/157,888

(22) Filed: Jun. 20, 2005

(51) Int. Cl. A61F 2/28

(52) U.S. Cl. 623/17.17; 623/23.63; 128/898

(57) ABSTRACT

The bone inferior to the maxilla sinus cavity of a patient is augmented, irrespective of the physical integrity of the Schneiderian membrane, by a resorbable package or vessel that contains granularized bone. The package is matched to be resorbed at about the time the desired bone augmentation is achieved. If an entry is available in the vessel, the ratio of the largest dimension of the vessel entry to the length of the vessel is less than 0.50. The resorbable package may be used with the Caldwell (window) technique or the osteotome technique.

### FIELD OF THE INVENTION

This invention relates to surgical apparatus and procedures for augmenting bone in the human jaw through bone growth as needed for installation of dental implants and, more specifically, to enhancing implant site bone depth in the maxilla bone, particularly in the event there occurs an unintentional break in the Schneiderian membrane that borders the sinus cavity.

### BACKGROUND

The dental implant is a well established, reliable and, perhaps, somewhat pricey technique for replacing a lost tooth in and/or anchoring dentures to a person's jawbone. The implants serve as a mounting for a prosthetic in the shape and color of the tooth that is being replaced or as a mount for a denture or bridge. Implants are designed to be screwed into the human jawbone and, due to bone integration that follows, becomes firmly anchored to the bone. Installation is permanent. The implant does not require the same care or periodic adjustments associated with bridgework and dentures.

The implant is typically formed of titanium, a metal, and generally is of a cylindrical shape. It contains a lower portion, that has a standard or self-tapping screw configuration, and an upper portion that has a post with a mount, such as a threaded hole, for attaching an implant prosthetic. The implant is installed during a surgical procedure in which a properly positioned hole is drilled into the jawbone at the location of the missing tooth (or the desired location for one of the multiple denture or bridge supports) and the implant is screwed into the hole. After installation, the jawbone grows over time and tightens its grip on the implant, eventually producing a strong permanent anchor to ones jaw. Thereafter a prosthetic, such as a false tooth that is shaped and colored to conform to the original tooth, is attached to the implant by means of a cooperative mounting, such as a socket located on the top of the implant.

An implant, typically, is between 8 to 16 millimeters in length and, is typically 3.3 to 5.0 millimeters in diameter. The width and depth of the jawbone at the intended implant site (or sites) must be wide and deep enough to accommodate the implant and must also possess adequate strength to hold the implant in place without fracturing the jaw bone. The bone depth at the implant site should be at least a few millimeters greater than the length of the implant. Hence, the bone depth should be between 10 to 13 millimeters at that site, depending on the length of the implant chosen for the installation. That depth is not available in every patient.

As a first step to any dental restoration procedure, thus, an x-ray is taken of the region to allow determination of the bone depth that is available and determine the amount of any insufficiency. It has been found in practice that a large number of persons, perhaps 30%, do not have sufficient bone depth capable of supporting an implant in the upper jawbone. The reduced bone depth is generally found in the region of the molar and pre-molar teeth in the upper jaw underlying the maxillary sinus. The insufficiency in bone depth may come about for any number of reasons. As example, if a tooth is extracted and not replaced or restored, the bone at the site of the excised tooth recedes, eventually thinning the bone at that region. Also a bacterial infection in and/or under the tooth that was lost, not only may destroy the tooth but eats away at the jaw bone, reducing the bone depth.

In order to install an implant in those circumstances, the depth of the bone at the site, if insufficient, must be augmented. The clinical reports of Boyne, James and Tatum early on recognized the need to supplement bone located inferior to the maxillary sinus to enable clinicians to perform alveloectomy surgery and subsequently place dental implants. Oral surgeons and periodontists have previously accomplished that fit, most recently with bone grafts and like procedures.

The principal advantage of the present invention is for bone augmentation in the region of the molars and pre-molars in the upper jaw and inferior to the maxillary sinus.

Two proven procedures for increasing bone depth in the maxilla sinus in the region of the molars and premolars are currently known and found in practice. The first is known as the Caldwell Technique or, alternately, as the Window technique. Both names may be used interchangeably in this specification. The second procedure is known as the Osteotome technique, the osteotome being a round dental chisel without a bevel that is used for cutting and expanding bone. Both techniques involve increasing the bone depth at least on the bottom of the maxilla sinus, which basically reduces the size of the sinus cavity as defined by the Schneiderian membrane, next discussed. Two text books that describe the foregoing restorative procedures in great detail for training of doctors are The Sinus Bone Graft, Ole T. Jensen, DDS, Quintessence Publishing Co. Inc, copyright 1999 and Practical Implant Dentistry, Arun K. Garg, D. M. D, Taylor Publishing Co. ISBN 0-9648918-1-6 to which the interested reader may make reference.

The sinus cavity in the skull is an air filtration space overlying the position of the molars and pre-molars that ranges in size from approximately ten to twenty milliliters of volume, depending on the individual. There's one such cavity on each side of the skull. The sinus cavity is bordered by the sinus membrane, also known as the Schneiderian membrane, a mucous membrane lining the nasal chambers. Both terms may be used herein interchangeably to identify the membrane. The sinus cavity has the capacity to pneumatize or crenate because of the supporting and surrounding conditions, such as loss of teeth or chronic irritation, and, yet, functions in relative health. Thus, the reduction in the size of this cavity, when moved superiorly, as by inserting bone material beneath the sinus membrane and raising the height of that membrane above the bone, does not pose a serious threat to the patient's health.

However, if the sinus membrane is damaged, bacterial contaminants are introduced into the cavity, resulting in inflammation and infection. Should such damage occur, all augmentation and implant procedures are halted until the damage is cured, which could take months. The membrane is a body part that is capable of auto-repair and, left undisturbed, the body repairs the membrane. That autogenuous repair takes four to eight weeks on average. Minor tears may also be fixed by applying a collagen tape across or along the tear.

In the Caldwell or window technique, as variously called, an osteotomy is performed on the lateral wall of the maxillary sinus above the tooth line by cutting a flap from the covering skin or gum and folding the flap out of the way to expose the bone. Then, using a small round bur in an electric rotary instrument, the outline of a window, an elliptical window as example, is ground into the lateral bone wall. Using that outline as a guide, the burring continues along the outline to a depth in the bone at which one is able to see the dark inner lining of the sinus membrane. The sinus membrane is rather fragile. Hence, care must be taken to avoid tearing the membrane with the rotary instrument.

With the connection to the cut-away bone portion weakened by the grinding, the formed window may be infractured from the maxillary using a few light taps with a mallet. Use of excessive force to infracture the external wall of the sinus cavity, however, may result in producing a large tear in the sinus membrane. That tear could compromise the success of the procedure. Despite the exercise of due care under the circumstances, because of the uncertainty in the bone wall thickness, the sinus membrane may inadvertently be torn or cut in perhaps twenty-percent of the cases. Thus, a principal purpose of this invention is to permit bone augmentation using either of the two procedures, slightly modified, even in the presence of a tear in the sinus membrane. As an advantage, the invention avoids the delays previously required to fix the tear in the membrane.

After opening the window in the maxillary sinus, the sinus membrane is gently pushed away from the inside wall of the sinus cavity in the region about the edge of the window and below that window using a curette instrument to lift the membrane away from the bone. That action opens and allows access to a portion of the sinus cavity in the region that overlies the desired implant site in the pre-molar and molar teeth line, at which one wishes to increase bone depth. That region is conducive to new bone growth using various types of bone augmentation materials. The open window is then packed full with bone granules and/or other bone graft material.

Typically, the best (and most sterile) bone granule material is that which is supplied from the bones of the patient. As example, the bone panel that was removed from the skull to form the open window in the Caldwell procedure is placed in a grinder and is ground to produce bone granules, granularized bone, which is the best form of bone to use for a bone augmentation process. Those granules are mixed with some of the patients blood and some additional bone granules supplied by a vendor of medical supplies to form a sludge-like mixture. If one does not wish to harvest bone material from the patient, then the requirements may be met by purchasing those granules from third party vendors, who are available to supply bone granules harvested from the bones of cattle, which may be used in human application. Those animal bone granules are sterilized by the vendor and may be used to form the blood and bone mixture. The mixture is then inserted into the formed window in the maxilla overlying the implant site using an appropriate dental spatula. The open window is also filled. The skin flap, earlier folded out of the way and stitched in place, has the stitches removed and is then folded back over the window and stitched closed to hold the particulate mixture in place under the light compression produced between the skin flap and the sinus membrane.

Bones are not static parts of the human body. The bones contain cells, nutrients, and blood. In a healthy person they can grow. It is found that within a period of approximately four to nine months, the bone granules placed in the sinus window solidify together and join to the surrounding bone. Through that growth the bone overlying the implant site attains at least the proper height (or depth). Thereafter the regular implant installation procedure may be administered to the patient, the details of which are known to those skilled in the art, are not material to the present invention, and, hence, need not be further described.

The second augmentation technique is next addressed. The Osteotome technique for bone growth in the upper jaw, also called sinus elevation, involves lifting the sinus membrane over the intended osteotome site of an implant using a number of osteotome. Starting with a small size (e.g. diameter) osteotome and continuing with a number of increasingly larger diameter sizes, a partial passage is formed though the ridge at the intended implant site. This technique may be used when some bone is available in a position on the ridge of the upper jaw, where the tooth was removed, below the sinus cavity. The shortfall in bone depth at the intended implant site is determined by an appropriate X-ray or CT scan.

Initially, the gum is cut along the ridge in the region of the intended implant site to form a flap, and the flap is flipped over out-of-the-way and stitched in place. That leaves the bone on a portion of the ridge exposed. The exposed area is washed with a water spray to remove remnants of blood and other debris from the area. The bone is scrapped to clean the implant site, and a hole into the ridge is drilled at the intended implant site to a depth that falls slightly short of drilling through the opposite wall as could breach the sinus cavity. The depth of the hole is frequently checked with a gage and if sufficient, a larger diameter drill is used to widen the drill hole initially formed. Widening is continued with additional drills of increasingly larger diameter. When the proper diameter is attained, an osteotome is inserted in the hole and is lightly tapped with a mallet to fracture the opposite surface of the bone.

A small disk-like piece of bone is detached by the fracture. Being forced outwardly by the mallet tap, the piece of bone so detached pushes against the abutting sinus membrane, and lifts the membrane from the bone in the region overlying the intended implant site. Ideally the foregoing result is attained without breaking the membrane. The osteoteme is removed. Then a mixture of bone granules and human blood is placed through and into the hole and pushed through into contact with the sinus membrane overlying the implant site. The constituents of the mixture and the origin of that mixture may be any of the blood-bone mixtures and origins earlier discussed in connection with the Caldwell technique, and need not here be repeated. The skin flap is restored in position and again stitched in place covering the open end of the passage to prevent loss of bone granules.

In one approach, the formed hole is completely filled with the mixture and allowed to integrate over a four to eight week period. Should that approach be selected by the surgeon or periodontist, the implant is not be installed until some months later, after sufficient bone integration has occurred. At that later time, the gum ridge (e.g. maxillary ridge) is again cut and flapped, the implant hole in the gum ridge is selected and drilled for the implant, the implant is installed, and the gum flap restored in position and stitched in place so that the bone will ultimately integrate with the implant. This is the more conservative approach to the implantation.

The favored approach, however, is to refrain from completely filling the hole with the bone granule mixture, and immediately install the implant into the hole, screwing the implant into place in the bone granule mixture, whereby the distal end of the implant reaches and abuts the bone granules earlier inserted as well as with some granules that move up in the hole alongside the implant. The gum flap is then restored in position and stitched in place. After four to eight weeks the bone growth joins the granularized bone mixture, the graft, to the maxilla bone and concurrently secures the implant in place. Installing the prosthetic on the implant follows, but not being directly related to the invention need not be described as part of this background.

The goal of sinus lifting surgery is to lift the Schneiderian membrane superiorly to allow bone grafting of the inferior aspect of the maxillary antrum and every effort should be made to minimize membrane perforation during that surgery. Because of the anatomy of the membrane or the antrum, perforation avoidance is not always possible. Perforations that are greater in size than 5 mm² pose a different problem. Large membrane perforation can allow the spreading of bone fragments within the sinus, leading to inflammation or infection, especially if particulate grafting material is used.

The foregoing bone augmentation techniques are proven. The problem arises only when the sinus membrane is torn or perforated, which occurs in about twenty percent of the procedures. That tear or perforation is determined easily with a negative pressure test. For that test the patient is instructed to take a deep breadth and while squeezing the patient's nose closed with the patients fingers, attempt to exhale. Normally, one can't exhale because there's no open path to permit the air to exit. However, if there's a tear in the sinus membrane, the air will be blown out the sinus cavity through the mouth, either through the open window that was cut-out for the augmentation procedure, if the first grafting procedure was used, or through the passage formed through the gum ridge, if the second grafting procedure was used. If that occurs, with one exception, the procedure is halted and the membrane is permitted to repair itself, heal, typically over a period of eight week. Only thereafter, may the procedure be resumed.

The exception is the augmentation procedure that is reported upon by author V. Fugazzotto, in an article entitled “Innovations in Periodontics,” Periodontal, vol. 74, no. 10, October 2003, pp1535-1540. When, during the course of the Caldwell bone augmentation procedure of the maxillary sinus, a serious perforation is found in the Schneiderian cavity, as would normally halt the procedure, Fugazzotto, describes continuing with a modified procedure. That procedure is to fashion a shallow pouch, containing a peripheral rim, out of a bioresorbable material, such as collagen. To some, that pouch, as shown in the article, resembles a shallow soup bowl in shape. That bioresorbable membrane is placed over the ostectomy site and may vary in size from 15 to 35 millimeters. The illustration in the cited article appears greater than life-size, and, hence, yields a larger than true-size dimensions for the membrane, if measurements are taken directly from the illustration. However, the ratio of those dimensions would appear correct, notwithstanding. As seen in the illustration, the maximum dimension to the entry to the formed pouch in Fugazzotto article is the same as the length of the pouch yielding a ratio of 1.0. The sinus membrane is elevated with care as before to release the membrane from the bone surrounding the formed window. The collagen pouch is pressed into and closely fits into the formed window and the rim abuts and is tacked to the outer surface of the bone surrounding the window. In this position, the pouch extends inside the sinus cavity overlying the bottom of the cavity, the location at which bone augmentation is to occur. Effectively, the collagen pouch is stood on its side. Then the bone granule and blood mixture is scooped up a bit at a time from its mixing bowl using a dental palate and is spread out into the collagen pouch. The bone-blood mixture continues to be dispensed into the pouch in that way until the pouch is completely filled.

The skin flap that was folded out of the way and stitched there in the initial step of the Caldwell procedure, as earlier described in this background, is released from the stitches. The flap is then returned to its original position covering the area of the formed window, and, hence, now also the open end of the pouch. The skin flap is then stitched in that location to hold the resorbable pouch in place and prevent the upended contents of the pouch from spilling out. The report's author indicates that bone growth occurs over a period of four to eight weeks using the bone mixture while the pouch is resorbed over that period. Only thereafter is the patient ready for the installation of an implant.

The foregoing procedure presented by Fugazzotto appears to avoid delaying the bone augmentation procedure, an obvious advantage, but requires personnel to fabricate a proper size bowl shaped appliance from resorbable material on-the-fly, so to speak, is limited in application to the Caldwell (e.g. window) procedure and not to the osteotome procedure, and, even as applied in the Caldwell procedure, appears to allow the bone mixture to be placed only in a limited area of the formed window. The doctor may not be able to place the bone mixture inside the cavity, say posteriorly, where some bone augmentation may be needed. Hence, the Fugazzotto approach has some significant limitations. As an advantage the vessel of my invention when used in the Caldwell procedure is not so limited and may be moved around inside the cavity and positioned where bone augmentation is needed.

Accordingly, a principal object of the invention is to provide a bone augmentation procedure for the sinus cavity of a dental patient that may be successfully performed even when the patient's sinus membrane is torn or perforated.

A further object of the invention is to provide a bone pack that is useful as a bone augmentation appliance for the sinus cavity of a dental patient.

Another object of the invention is to eliminate the problem and delay caused by the occurrence of a torn sinus membrane during a maxilla sinus bone augmentation procedure, irrespective of whether the procedure is of the Caldwell type or the osteotome type.

A further object of the invention is to provide bone graft appliances, which I sometimes call pillows and sleeves, useful in implementing a sinus graft procedure, particularly when the sinus membrane is torn or perforated during the procedure.

An additional object of the invention is to streamline and improve the efficiency and universality of bone augmentation of the inferior of the maxilla sinus cavity by making the procedure independent of the integrity of the Schneiderian membrane.

The foregoing and additional objects and advantages of the invention, together with the structure characteristic thereof, which were only briefly summarized in the foregoing passages, will become more apparent to those skilled in the art upon reading the detailed description of a preferred embodiment of the invention, which follows in this specification, taken together with the illustrations thereof presented in the accompanying drawings.

### SUMMARY OF THE INVENTION

Bone augmentation is often needed for a dental patient to enable installation of a dental implant. A surgical dental graft appliance augments bone inferior to the maxilla sinus cavity, irrespective of the physical integrity of the Schneiderian membrane, by a combination of a non-metallic vessel of resorbable material, one that is porous to human bone cells and blood cells, and granularized bone augmentation material included in that vessel. With the vessel being at least partially filled with the granularized bone augmentation material and placed at a location at which bone augmentation is desired, the granularized bone augmentation material over a substantial period of time grows to the patient's maxillary bone inferior of the Schneiderian membrane while the vessel is concurrently gradually resorbed within the body. In a specific aspect to the invention, the resorbable material is formed of collagen and the granularized bone augmentation material is a mixture of bone granules and human blood that is compatible with the patients blood type. Preferably, for minimum rejection, that blood is harvested from the patient.

To apply the invention, both the Caldwell (window) maxillary sinus bone augmentation technique and the osteotome technique are both modified to incorporate the bone graft appliances of the invention.

As an advantage even when the sinus membrane remains unbroken, the availability of the present bone graft appliance opens the field of dental implants to dental workers who may possess lesser skill or who worry to much and are more likely to produce a cut in the Schneiderian membrane when performing either sinus bone augmentation procedure. With greater numbers of persons entitled to practice bone augmentation, dental patients should benefit by lowering cost due to the availability of greater numbers of suppliers. A second factor is that the invention provides a low cost insurance that the new bone augmentation procedures provide for even the most careful surgeon or periodontist

As an advantage the invention obviates the sinus membrane as a restrictive factor in the implementation of maxillary sinus bone grafting procedures. Tearing of the membrane need not be considered as a material factor in the bone augmentation procedure.

The foregoing and additional objects and advantages of the invention, together with the structure characteristic thereof, which were only briefly summarized in the foregoing passages, will become more apparent to those skilled in the art upon reading the detailed description of a preferred embodiment of the invention, which follows in this specification, taken together with the illustrations thereof presented in the accompanying drawings.

### BRIEF DESCRIPTION OF THE DRAWINGS

In the drawings:

FIG. 1 is a pictorial of a first embodiment of the resorbable vessel useful for augmentation of bone in the maxilla cavity inferior using the window sinus graft surgical procedure;

FIG. 2 is section view of the embodiment of FIG. 1 taken along the lines 2-2 in FIG. 1;

FIG. 3 pictures a second embodiment of the invention for the window sinus graft procedure;

FIG. 4 pictures a fourth embodiment for application in the window sinus graft procedure;

FIGS. 5A and 5B picture a fifth embodiment for the window sinus graft procedure;

FIGS. 6A and 6B show a sixth embodiment of the invention useful in the window sinus graft procedure;

FIG. 7 is another embodiment of the invention useful in the osteotome sinus graft procedure;

FIG. 8 is a pictorial showing an initial step in buring a window into the maxilla sinus cavity during a Caldwell-type bone augmentation procedure, and is used in explaining the method of applying the preceding embodiments;

FIG. 9 is a pictorial of another stage in the bone augmentation procedure in which bone covering of the window is removed to form the open window in the maxilla sinus cavity and expose the Schneiderian membrane;

FIG. 10 is a pictorial illustrating installation of the vessel of FIG. 6A into the maxilla sinus cavity during the Caldwell-type bone augmentation procedure;

FIG. 11 is a pictorial illustrating installation of the vessel of FIG. 4 into the maxilla sinus cavity during the Caldwell-type bone augmentation procedure as an alternative to that illustrated in FIG. 10;

FIG. 12 is a pictorial of a section of the maxilla showing the maxilla sinus cavity and tube-type bone augmentation vessels useful in the discussion of a osteotome-type bone augmentation procedure;

FIG. 13 is an enlarge portion of the illustration of FIG. 12 intended to show additional details;

FIG. 14 is a section of FIG. 12 taken along the lines 14-14 in FIG. 12; and

FIG. 15 is a pictorial view of another osteotome type procedure in which the implant is installed concurrently with the tube-type bone augmentation vessel.

### DESCRIPTION OF THE PREFERRED EMBODIMENTS

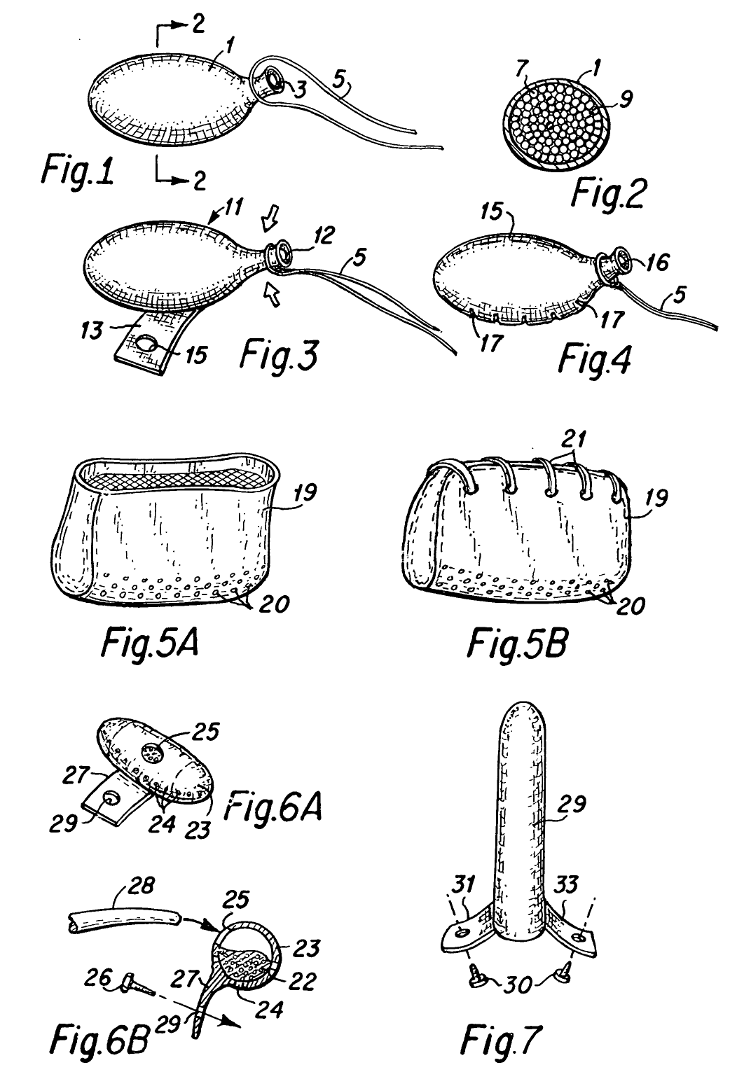

In the embodiment of FIG. 1 to which reference is made a vessel 1 is formed of walls of a bio-resorbable material that is porous, allowing bone and blood cells to pass into the vessel from the exterior. The only material presently known to applicant to fit the foregoing characteristics and function is a collagen. That material constitutes the fibrous protein constituent of bone, cartilage, tendon, and other connective tissue; and is harvested, as example, from human cadavers or from animals. It is converted into gelatin by boiling. Vessel 1 includes an entrance 3 located at the restricted distal end. As illustrated in the section view of FIG. 2, the contents of the vessel comprises a mixture of bone granules 7, which are solids, and human blood 9, a liquid; and the mixture fills the vessel. Returning to FIG. 1, a bio-resorbable thread 5 is stitched through entrance 3 to squeeze the entry 3 closed and block the contents from spilling out. Entrance 3 is quite small relative to the length of the vessel, an entrance of about 4 mm and length of about 48 mm for a ratio of abut 0.08 there between in the figure

As one will appreciate, the term vessel is used in a very generic sense as something that has the capability to hold a mixture but allows seepage through the pores of the walls, and may be uncapped. For that reason the vessel in the context of this invention may encompass or subsume like terms such as, but not limited to, container, pouch or bag. Where the pouch has the appearance of a pillowcase, one may even refer to vessel 1 by that term. In addition, where the resorbable pervious vessel or bag is filed with the granularized mixture the resultant product may be said to define a somewhat special “bag of bones.”

Because the material of the vessel is porous to bone cells and blood cells, the bone cells and blood cells may migrate through the wall material of the vessel. When the bone granule filled vessel is placed against a person's bone, a living substance, the external bone effectively migrates to the bone granules and blood inside the vessel, and produces integration using the bone granules as a principal ingredient of that new growth. The growth occurs slowly. Typically, it is found that about ten to fifteen millimeters of bone growth is completed by the human body in approximately four to nine months time.

Because the material of the vessel walls is bioresorbable, when the vessel is placed inside the environment of the human body, the material gradually dissolves away, a process referred to as resorbtion. That resorbtion process generally occurs slowly, measured on the order of weeks, not days. By design, the physical characteristic of the resorbable wall material that is relevant to the speed of resorbtion is the wall thickness. The wall thickness is ideally chosen to possess a resorbtion time that matches or slightly exceeds the period at which the desired bone growth is completed. Ideally, one should have the bone material completely taken up into the bone growth and have the vessel disappear through resorbtion concurrently. That involves some engineering and testing development in optimizing the present invention, which will be accomplished in the future as the invention is implemented with practical embodiments in practice, not yet available.

In one embodiment of the invention, the vessel may have thin limp walls, like a paper or plastic bag, so that when the bag is empty, the vessel walls collapse in volume, but, when the vessel is filled with the bone granules, the vessel expands to the larger volume illustrated in the figure. In an alternative embodiment of the invention, the vessel may have stiff or rigid walls that are self-supporting and retain the same shape and volume, even when empty, like a glass milk, or juice bottle, as would occur with relatively stiff or thick walls when the vessel is formed by molding or extrusion. Thinner walls of course resorb more quickly than thicker walls.

In the embodiment of FIG. 3 vessel 11 is essentially the same structure as vessel 1 of FIG. 1, containing walls of resorbable material and modified to include a tab 13 that extends from the side wall of the vessel. The tab contains a small hole 15 therethrough, a tack hole, through which one may insert a dental tack or screw to fasten to the maxilla bone. The vessel contains the small entrance 12, and that entrance is squeezed closed by the resorbable thread 5, the ratio of the size of the entrance to the length of the vessel is the same as the prior embedded version. The tab may be stitched to the vessel illustrated in FIG. 1 or, if the collagen material is molded to shape, the tab could be molded to the vessel wall. In this embodiment, the formed “bag of bones” may be fastened in place by tacking the tab to the maxilla bone. That prevents vessel 11 from moving about. The flap of skin that was initially cut open and stitched out of the way during the initial stage of the Caldwell procedure, later described, is then restored over the window in the maxilla sinus containing the filled vessel, 7 and stitched in placed, assisting to maintaining the bag of bone in place.

Reference is next made to FIG. 4, which pictorially illustrates a third embodiment of the bag of bones invention. A ellipsoidal or bladder-shaped vessel 15 is formed of the resorbable porous collagen material. That is manufactured slightly thicker and stiffer than the corresponding material used in most of the preceding embodiments and is self-supporting. Hence, the vessel retains the illustrated shape, even when empty. As in the preceding embodiments the distal end of the vessel is restricted in size and contains an entrance 16 at that restricted size end location. Small punctures or holes 17, only two of which are labeled, are punctured, cut or molded within the wall portion that is intended to be placed in abutment with the bone that is to be augmented by the window procedure, The vessel is filled with The mixture of granularized bone material and human blood is inserted through entrance 16 into the vessel, filling the vessel, in preparation for the dental procedure. The material forming the vessel wall is essentially porous to blood cells and bone cells, just as in the preceding embodiments, but the naturally formed pores in the material are not illustrated. However, the mechanically fabricated holes 17, which are illustrated, are smaller in diameter than the pores of the material and slightly smaller than the size of the bone granules. Those holes are thought to enhance the interaction between the bone granules in the vessel and the bone being augmented to more quickly commence bone growth. As those skilled in the art are aware, holes 17 may be mechanically formed by puncturing the material with the tip of a needle. That tip is about one-third of a millimeter in diameter. Resorbable thread 5 is stitched around the entrance to prevent the contents from spilling out, particularly during handling.

A still further embodiment of the periodontal bag of bones is pictorially illustrated in FIGS. 5A and 5B to which reference is made. In the foregoing construction the material that forms vessel 19 is extruded into the pillow of bag-like shape illustrated in FIG. 5A. The lower side and bottom portions of bag 19 contain perforations 20, only one of which is labeled. Perforations 20 extend through the wall and open into the interior. Then, the bag is filled with the mixture of bone granules and blood, not illustrated, and the bag is sewn closed using a resorbable thread 21 so there's no longer any entrance (or exit) to the vessel. The perforations 20 are intended to enhance the initial migration of bone and blood cells between the interior and exterior of the bag and, ideally, produce a faster start to augmentation of the bone at the position where the bag is positioned and in contact with the maxilla bone. The bag of bones thus formed is used in the same way for the same purpose as in the preceding embodiments.

FIGS. 6A and 6B, to which reference is made, shows an additional embodiment of the invention, respectively, in front perspective and in section. The vessel 23 of this embodiment contains semi-rigid walls that are self supporting and defines a container of a predetermined size and volume, irrespective of whether the container is filled or empty. The walls are formed of the resorbable material, such as collagen, that is pressed or otherwise molded in one or two pieces that are then joined together by heat and pressure (or an edible adhesive) to form the ellipsoidal shaped geometry illustrated. An entry 25 is a hole located in the central portion of the body. The entry hole is large enough in size to admit entry of the spout 28 of a dispenser that dispenses the granularized bone and blood mixture into the interior of container 23. The lower side and bottom portions of the wall include a large number of minute perforations 24, only one of which is labeled, that are slightly smaller than the size of the bone granules. This embodiment also contains an outwardly extending tab 27 attached to the side of the ellipsoidal body. That tab contains a screw hole 29 through which a screw 26 is able to attach the assembly to the patients skull to hold the assembly.

As shown in the side section of FIG. 6B, the granularized bone mixture 22 is deposited in the interior and needs only to partially fill the defined chamber in vessel 23. The entry 25 is located so as to be accessible from the exterior of the body (and not oriented so as to face into the sinus cavity where the entrance would not be accessible and could not receive the dispensed bone granules. The perforations 24 are intended to enhance the initial migration of the bone and blood cells between the interior and exterior bone. The perforations are preferably included. This is the same function previously described for the corresponding perforations 17 and 20 in the embodiments of FIGS. 4 and 5B.

Given the teachings of the present invention and additional development by the informed skilled person, additional vessel designs and constructions containing resorbable walls are certain to be created in the future that will accomplish at least the same function as described herein. All of those additional structures of necessity incorporate the present invention. In practical embodiments of the foregoing vessels, the resorbable walls may be constructed of collagen that is about one millimeter thick. the respective vessels may be fabricated in advance and inventoried in sizes of 15 mm length by 4 mm height; 17 mm length and 6 mm height, 18 mm length and 7 mm height and so on so that the periodontist has a suitable size available when needed.

For the osteotome technique, a vessel of a different shape is required. As pictorially illustrated in FIG. 7, vessel 29 is of an elongate tubular geometry with a closed distal end and open on the other. The tubular walls are constructed of a resorbable material, such as collagen, that is porous to blood and bone cells. The walls are also thick and stiff enough to be self-supporting to maintain the tubular shape of the vessel even when empty. In some respects the geometry of the container resembles a test tube or a sleeve. The open circular proximal end of the vessel 29, not visible in this view, contains two radially outwardly extending tabs 31 and 33 located at the open end that extend along a common diameter. Vessel 29 is suited for placement inside a hole in the jaw, and is tacked in place using the tabs, which is described in greater detail in the succeeding paragraphs. A mixture of the granularized bone material and human blood, earlier described, but not illustrated in this view, is packed in the vessel 29 as in the prior embodiments at least partially filling the container.

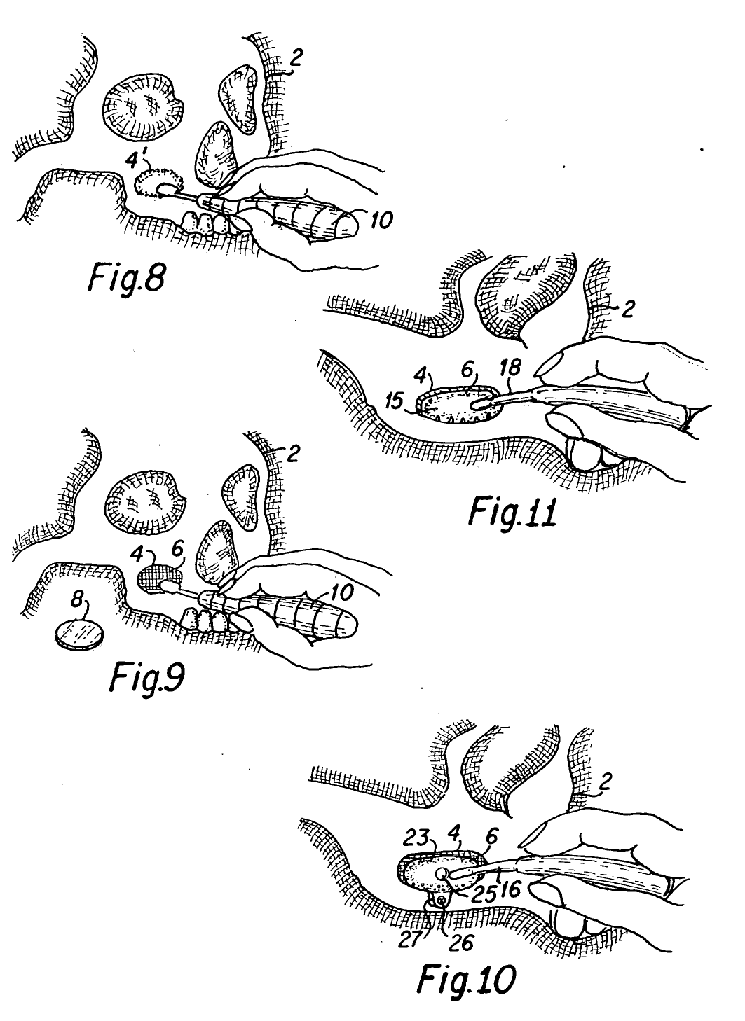

The application of the embodiments of FIGS. 1-6B in the Caldwell window procedure is next described and is followed by the application of the embodiment of FIG. 7 in the osteotome procedure. Before proceeding further, the reader may wish to review the description of the Caldwell (windows) technique earlier presented in the background section of this specification. Reference is made to FIG. 8, which depicts a portion of the patients skull 2, and the outline 4′ cut into the skull with the burring tool 10 to define the portion of the skull that's to be removed to define and form the open window into the sinus cavity, shown in FIG. 9 as window 4. For clarity of illustration, the patient's flesh has been removed from the skull, but, in practice, remains on the person, and during the procedure, the upper lip portion surrounding the patient's mouth must be held out of the way to allow the surgeon access to the elevated position in the upper jaw, where the window is formed. Window 4 that is cut into the maxillary sinus to allow access to the Schneiderian (sinus) membrane 6, and the sinus cavity (defined by that membrane) is illustrated in FIG. 9. A cutout portion 8, the removal of which defined open window 4, is also shown intact as a single piece. In practice, however, the cut-out portion is usually broken into multiple pieces.

A smaller section of skull 2 is illustrated in FIG. 10 in slightly larger scale than presented in FIGS. 8 and 9. As shown in FIG. 10, bone vessel 23 (of FIG. 6A) is placed through window 4 and to partially fit inside the sinus cavity in abutment with and in inferior position underlying Schneiderian (sinus) membrane 6. As one appreciates, the bag of bones occupies some space inside the sinus cavity underneath the Schneiderian membrane, raising that membrane and, thereby reducing the size of the sinus cavity defined in the chamber by that membrane. That reduction remains even after the bone augmentation is completed, since the purpose of the augmentation is to increase the depth of the bone in the jawbone inferior to the sinus membrane. That bone growth occurs upward into the sinus cavity and incorporates the bone granules in the vessel.

Assume that bone augmentation is to be implemented on the right side of the patients maxillary sinus bone, as viewed by the patient. That is the position of the skull depicted in FIGS. 8-10. As shown, the premolar and molar teeth on the right side are missing. The patient desires to install one or two implants in place of the missing teeth. By means of an x-ray or CT scan the periodontist determined that the upper jawbone was too thin in that region. Before it is permissible to install an implant, the bone in the sinus cavity, overlying that location requires augmentation. For this discussion the Caldwell technique is selected as the procedure for the bone augmentation. Once the bone augmentation is performed on the patient a period of about four months is required to produce sufficient bone growth. Preferably, the implant will be installed (and cosmetic procedures will be added) later, only after the augmentation is completed.

In the Caldwell (or Window) procedure the patient's mucosa and gum skin is pulled back and held out of the way to the maximum extent, exposing the gum line and the skin superior thereto covering the maxilla. An incision is made in the ridge of maxilla above the gum line, not illustrated, and the oral mucosa skin is cut to form a flap, also not illustrated. Using a curette, the oral mucosa is cut and elevated from the bone to fully release the flap, thereby exposing the underlying bone of the maxilla shown in FIG. 8. The skin flap is then pulled back using a clamp and stitched in place to keep the skin flap out of the way, leaving full access to the underlying maxilla bone. A wash, not illustrated, is used to clean blood and other debris from the exposed bone. Using a rotary bur in rotary tool 10 the outline 4′ of window 4 is cut into the maxilla bone. The cut is deepened along the outline until the dark blue color of the Schneiderian membrane 6, the sinus membrane located behind (and under) the window 4 region, bordering the sinus cavity, is visible. Referring to FIG. 9, once that stage of burring is reached, a few careful light taps on the cut-out portion 8 of the bone with a dental bone tamper, not illustrated, and the bone panel fractures from the maxilla, breaking away in one or more pieces, and forming window 4. Using a tong, not illustrated, the panel piece (or pieces) 8 of the bone are picked out and removed to a safe off-patient location.

Those removed pieces of bone can be granularized and then incorporated later in the augmentation operation. Using a dental elevator or sinus curette, not illustrated, to reach through the window 4, the periodontist or surgeon carefully pries the membrane 6 away from the inside wall about the periphery of window 4 and lifts the portion of that membrane that lies below the window off the floor of the sinus cavity, exposing the inferior region of bone.

Ideally and with careful moves by the surgeon, the foregoing stage in the Caldwell procedure is accomplished without tearing Schneiderian membrane 6. As earlier discussed, such is not always the case, which was a motivating factor for creation of the present invention. Assume next that membrane 6 was torn. As a consequence, for one, it then becomes possible for bone chips to enter the sinus cavity and cause an infection. Hence, at this stage, the prior procedure was to halt the augmentation procedure, and embark on restoring the membrane, postponing augmentation to another time. Not so with the present invention. Alternatively, at this stage the Fugazzotto technique described in the background could have been used to permit the augmentation procedure to continue. But for reasons given, the latter procedure is not undertaken. Instead, an appliance according to the present invention is chosen.

Reference is again made to FIG. 10. The length and width of window 4 is measured and approximate dimensions determined. Given those dimensions, a resorbable bone pack or vessel of the appropriate size is selected from an inventory of those dental appliances maintained in various sizes under sterile conditions. Alternatively, an empty vessel of the appropriate filled size is taken from an inventory of various size sterile bags. Any of the embodiments earlier described in connection with FIGS. 1-6B may be used for the Caldwell procedure, and at present, no one embodiment is known to be better than any other embodiment. For this description, the resorbable bone vessel selected is one which is vessel 23, illustrated in FIGS. 6A and 6B.

Using a tweezer, not illustrated, the resorbable vessel 23 is deposited inside window 4 and is seated on the inferior wall of the sinus cavity overlying the implant site and in abutment with an unbroken portion of the Schneiderian membrane 6. Further, the vessel is tacked to the bone via tab 27. Then vessel 23 is filled with a mixture of human blood, granularized bone and granularized allograft bone (i.e. bone taken from another human) or exenograft bone (i.e. bone taken from an animal). the blood should be a universal type to avoid an adverse reaction by the patient's immune system. Preferably, the blood is swabbed from the patient in the foregoing stages of the operation. That avoids use of the blood of a third party that might be rejected by the patient's immune system and cause an adverse result. The granularized bone of the patient may be obtained by granularizing the extracted bone 8 (FIG. 9), or extracting bone from another site on the patient and granularizing that bone. Alternatively or additionally, artificial bone may be obtained from an outside supplier and used. The bone blood mixture is picked up by the dental bone carrier 16, pressed through entry 25 and deposited inside vessel 23, continuing until the vessel is filled. The stitches, not illustrated, that held the skin flap out of the way are then cut or removed, the flap is re-folded over window 4, covering entry 25 and vessel 23, and stitched in place. That skin flap holds vessel 23 in place in the portion of the sinus cavity. The four-month waiting period then begins.

As one appreciates, it is not possible for bone granules to access the sinus cavity behind the Schneiderian membrane as could lead to an infection for the patient, since the granules are contained in a vessel that is essentially closed and from which the particles cannot escape. With the invention, the bone augmentation procedure continues, a decided advantage of the invention that is shared with the Fugazzotto approach, earlier described.

Now, returning to the stage in the procedure in which has determined that the Schneiderian membrane remains unbroken, one may nonetheless follow the same procedure described for handling the bone augmentation described in the preceding paragraphs when the membrane was found to be broken. By following the same procedure and using the resorbable bone vessel, the surgeon or periodontist in effect is buying insurance for both they and the patient. The added cost is small. But being freed from an overwhelming and distracting concern over a possible or undetected break in the membrane occurring in the following stages as would halt or otherwise complicate the augmentation and/or implant procedure, the surgeon and periodontist knows that the augmentation procedure can be completed, notwithstanding any such tear.

Reference is made to FIG. 11. This figure shows the same pictorial view of a portion of the maxilla bone that was illustrated in FIG. 10, but in which the resorbable vessel 15 embodiment of FIG. 4 is inserted through window 4 and into the sinus cavity. The entry and resorbable thread are packed inside the sinus chamber and are not visible in this view. Vessel 15 was previously packed with the mixture of bone granules and blood, whereas in FIG. 10, vessel 23 with stiff self-supporting walls was filled only after installation in the sinus cavity. A dental spatula 18 may be used to assist in pushing the vessel into place. Once that is completed, the skin flap is released from the stitches that held the flap out of the way and the flap is re-folded into the original area where window 4 is now located, and covers the window and the vessel 15, holding the articles in place. As in the prior embodiment, the patient begins the waiting period of four to eight months during which time the bone augmentation occurs. The bone and blood cells migrate through the walls of vessel 15 and the bone grows. Concurrently, the walls of the vessel are absorbed in the body.

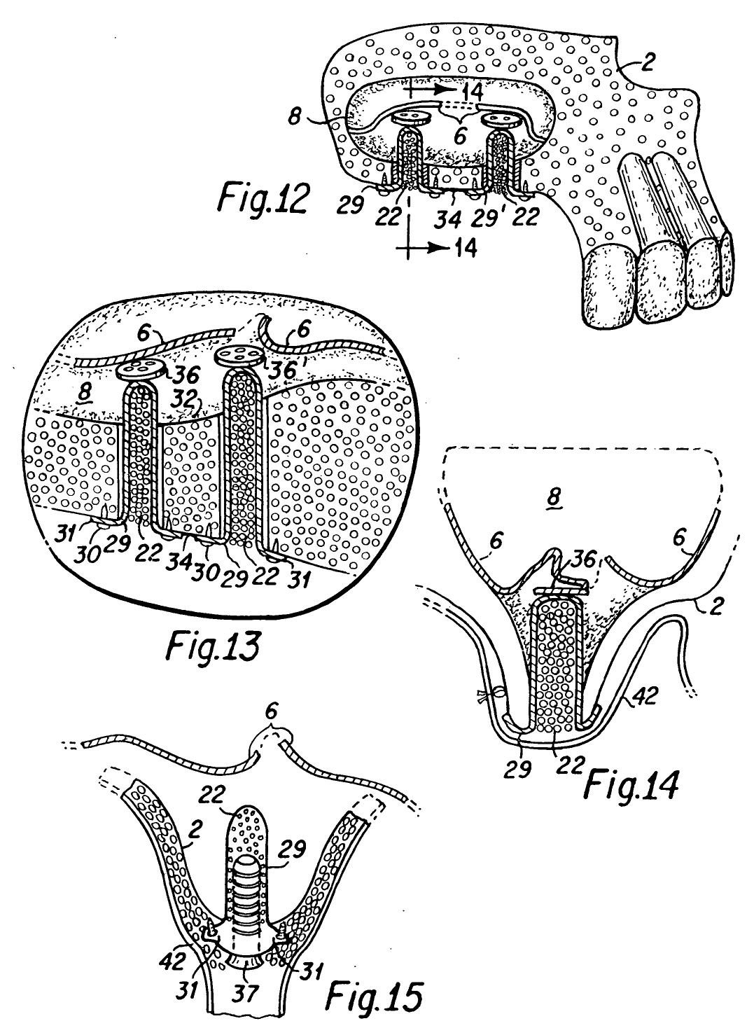

The sleeve-like or tube-like shaped resorbable vessel 29 of FIG. 7 is most useful in an osteotome procedure, a prior art procedure earlier described, that is modified to incorporate the vessel. Reference is made to FIG. 12 which pictorially illustrates in section a portion of the upper jaw, including maxillary sinus region 8, on which the osteotome procedure of bone augmentation was carried to the bone growth waiting stage; and in which two resorbable bone augmentation vessels 29 and 29′ have been installed. So as to avoid potential confusion, the reader is reminded that the maxillary sinus region 8 shown in section in the figure is not the “window 4” formed in the side wall of that region presented in the Caldwell procedure, earlier described. For clarity of illustration, the gum is omitted.

Initially, the reader may wish to review the information presented on the osteotome procedure in the background section of this specification. As shown a pre-molar and a molar were removed earlier. One or two of those teeth are now to be replaced by implants. Through use of the x-ray or a cat scan, the depth of bone in the sinus cavity in the upper jaw of the patient at the site overlying the missing pre-molar and molar teeth is measured and the insufficiency in bone depth at that site determined. Assuming, as before, that it is necessary to augment the bone in the bottom wall 32 of the sinus cavity 8, the maxilla sinus. In the osteotome procedure, access to the bottom wall 32 of the sinus cavity is made through the bone 34. The foregoing is more easily viewed in FIG. 13, which pictorially illustrates a portion of the bone and sinus cavity in an enlarged scale.

An incision of predetermined length is made along the ridge 34 to cut out of the skin on the ridge lingual (i.e. inside) and buccal (i.e. outside) flaps of skin, not illustrated, at the site of the missing molar and pre-molar teeth. The formed flaps are folded over and stitched in place so as not to interfere with the additional steps of the osteotome procedure. The exposed bone may be washed to cleanse the bone of blood. A drill bit of a predetermined diameter, typically seven to eight millimeters, is used in a rotary drill. Holes are drilled at each site into the ridge, but are not drilled entirely through. A depth gage is inserted from time to time into the drilled hole during drilling to check the depth of the hole and ensure that the drill does not accidentally extend through the opposite surface 32 of the sinus cavity, because that would permit the drill to perforate sinus membrane 6, which is not desired. When the depth measured is about one millimeter less than the depth of bone, drilling is halted. A bone tamper is then inserted in the drilled hole and tapped lightly with a mallet, neither of which is illustrated, to fracture the remaining millimeter of bone. That action, effectively breaks a disk shaped portion 36 (and 36′) of bone from the bottom wall of the sinus cavity and pushes that detached portion against the Schneiderian membrane 6 bordering the sinus cavity.

Using the osteotome, the membrane is carefully manually pushed upward to further detach portions of the membrane from the bone in the area adjacent to the newly formed hole. If 4 or 6 millimeters of additional bone height is needed for the augmentation, then one must ensure that the membrane is pushed upwardly by that amount. The foregoing is more clearly illustrated in the enlarged section in FIG. 13.

At this juncture the osteotome procedure departs from the prior art procedure in order to incorporate the invention. One selects from an inventory a test tube-like elongate cylindrical vessel 29 for one hole (and a like vessel 29′ for the second hole) formed of the resorbable collagen material. The diameter of the elongate tubular portion of the vessel is slightly smaller than the diameter of the drilled hole to permit the tubular member to easily be inserted through the drilled hole with minimal frictional interaction with the walls of the hole. The length of the vessel is at least equal to the depth of bone desired at the site of the formed hole and suitably of a greater length, incrementally equal to the depth of bone augmentation that is planned, such as is illustrated in FIG. 13, showing the distal end of the vessels to extend above the bottom wall 32 of the sinus cavity. To enable convenient access to vessels of an appropriate size without delaying the procedure, the surgeon or periodontist should likely maintain an inventory of such resorbable tubular vessels in varying lengths and diameters is maintained in a sterile environment or package.

Vessel 29 is inserted all the way into the drilled hole, whereby the distal end may be in contact with the lifted sinus membrane 6, directly, or indirectly, through the bone chip 36. Likewise vessel 29′ is inserted all the way into the second drilled hole, and the distal end of that vessel may be in contact with the lifted sinus membrane 6, directly, or indirectly, through the second bone chip 36′. For purposes of this description, assume that sinus membrane 6 remains intact at this stage as represented by the dotted line. Tacks or screws 30 are then placed through the respective tabs 31 and into the bone to hold the respective vessels in place.

The mixture of bone granules and human blood is prepared or otherwise obtained from a supplier. The mixture may be obtained in any of the ways earlier described in connection with the description of the preceding Caldwell procedure, which need not be repeated. Using a dental bone packer, not illustrated, portions of the bloody bone mixture are inserted into the vessel 29, while the patient has his or her head tipped backward, and, preferably, with the patient lying prone. The mixture is then gently tamped down. That procedure is repeated until the vessel is full. The foregoing granule filling is also carried out for the second vessel 29′.

Reference is made to FIG. 14, showing a section of the maxilla bone taken along the lines 14-14 in FIG. 12. In this view the vessel 29 is shown extending through the passage formed through the ridge of the jaw and extends into the sinus cavity with the distal end of vessel 29 underlying the Schneiderian membrane 6. The membrane 6 is shown torn. Although that membrane is torn, none of the bone granules 22 in vessel 29 have access to the perforation in the membrane and, hence, are unable to enter sinus cavity 8. To complete the procedure, the initial stitches in the gum skin flaps 42, that were stitched out of the way during the initial steps of the procedure, are released and folded back in place over the ridge and over the open end of vessels 29 and 29′, and is stitched in that position. The skin flaps cover the open ends of the resorbable tubular vessels 29 and 29′ to block escape of the confined bone granules and/or blood. Further, the internal friction between the bone-blood mixture 22 and the walls of the respective vessel 29, 29′ aids the skin flaps in preventing the escape of any of the mixture. The parties and patient now enter a period of waiting.

After a period of about four months, one finds that bone growth occurred on the inner surface of the sinus cavity and throughout the hole, effectively closing the formed passage through the bone, while the walls of vessel 29, 29′ have been resorbed by the patient's body. That completes the described embodiment of the osteotome bone augmentation procedure. Thereafter, installation of the implants and the prosthetic false teeth mounted to those implants is carried out in the normal way, just as if one were dealing with natural bone. Since those procedures are known in the art and are not necessary to an understanding of the invention they are not repeated.

In the foregoing osteotome procedure, the assumption was made that the Schneiderian membrane 6 remained intact during the entire procedure. However, assume next that the membrane was torn, perforated, at any stage in the procedure, as illustrated by the solid lines for the membrane 6 shown in FIGS. 13 and 14, such as occurs frequently in the prior art implementations of the osteotome procedure. If the bone granules that are necessary for the bone augmentation were not confined or restricted by the walls of the respective vessel 29, some granules could have passed through the tear in the membrane, entered the sinus cavity and caused an infection and/or other problems. For that reason, once a break in the membrane occurred in practice the augmentation procedure would have been brought to a halt, until the membrane repairs itself or was otherwise repaired. Because the bone granules were confined, however, the augmentation procedure continues unabated. The break in the membrane is of no effect. And while the bone growth occurs over the next few months to augment bone depth of the bottom wall of the sinus cavity, the membrane concurrently repairs itself, one of the miracles of the human body.

Even though waiting following the osteotome-type procedure is preferred as a conservative approach to implant surgery, the invention is not so limited. Those wishing to expedite the procedure, if that is found permissible without jeopardizing the patient, may in fact install an implant along with the tube-type vessel. That additional procedure is represented by FIG. 15 to which reference is made. In this figure, the elements of the skull and maxilla, and those of the vessel, which were described in the preceding embodiments are identified by the same number in this figure. As shown resorbable vessel 29 containing the mixture of bone granules and blood 22 is installed in the maxilla 2. The elongate tubular vessel extends through a passage in the ridge and into the region underlying the sinus cavity beneath the Schneiderian membrane 6, assumed to be torn, and the vessel is tacked in place to the bone by means of the outwardly extending tabs 31. In this variation of the procedure, an implant 37, that is slightly smaller in diameter than vessel 29, is centered in vessel 29 and screwed into the bone granule and blood mixture inside that vessel, screwing the implant to the requisite depth inside the vessel desired by the surgeon or periodontist. The skin flaps 42 are then stitched in place covering the end of the vessel 29 and implant 37. Now during the waiting period, not only does bone growth occur to the surrounding maxilla bone 2, but as the bone granules grow together, they solidly anchor the implant in the jaw. The implant becomes permanent and can't be removed without removing the surrounding bone. Concurrently, the walls of the vessel are resorbed by the body during the four-month wait. The foregoing procedure speeds up the process of dental restoration. Only through experienced gained in practice will one be able to determine which is the better procedure from the standpoint of patient health.

Referring again to the various embodiments of the bone vessel of FIGS. 1-7, one realizes that the embodiments do not contain an entry or contain an entry that is basically compressed in size or squeezed shut. It is seen that the ratio of the largest dimension of the vessel entry, if one exists, whether height or width or, if circular, the diameter, to the length of the vessel in my invention is very small, under 0.50. It would appear that such a ratio is a sufficient physical distinction to distinguish the word description of some embodiments of my invention from inadvertently describing the different and less versatile vessel of Fugazzotto. In a practical embodiment of the embodiment of FIGS. 1 and 3, the diameter as closed is 4.0 mm and the length of the vessel is 28 mm for a ratio of 0.14. For the practical embodiment according to FIG. 4 the diameter is 7 mm and a length of the vessel of 40 mm for a ratio of 0.17. For the embodiment of FIG. 6 the diameter of the entry in a practical embodiment was 5.5 mm and the length of that embodiment was 32 mm for a ratio of 0.17. And for a practical embodiment according to the embodiment of FIG. 7, the diameter of the entry was 4.0 mm and the length 18 mm for a ratio of 0.22.

It is believed that the foregoing description of the preferred embodiments of the invention is sufficient in detail to enable one skilled in the art to make and use the invention without undue experimentation. However, it is expressly understood that the detail of the elements comprising the embodiment presented for the foregoing purpose is not intended to limit the scope of the invention in any way, in as much as equivalents to those elements and other modifications thereof, all of which come within the scope of the invention, will become apparent to those skilled in the art upon reading this specification. Thus, the invention is to be broadly construed within the full scope of the appended claims.

What is claimed is:

1. A surgical dental graft appliance (bone augmentor) for augmenting bone inferior to the maxilla sinus cavity of a patient, irrespective of the physical integrity of the Schneiderian membrane, comprising in combination:
granularized bone augmentation material;
a vessel, said vessel being formed of a resorbable material that is porous to human bone cells and blood cells to permit migration thereof out of and into said vessel, said resorbable material being slowly resorbed by the patients body when held in contact with the patient's body fluids; and
said vessel being at least partially filled with and retaining said granularized bone augmentation material prior to resorbtion of said vessel by the patient;
said granularized bone augmentation material growing to the patient's maxillary bone inferior of said Schneiderian membrane over a substantial period of time while said vessel is concurrently being gradually resorbed within the body;
said vessel having a predetermined length and being closed or containing an entry, and, when containing an entry, the ratio of the largest dimension of said entry to said predetermined length being less than 0.50.

2. The surgical dental graft appliance as defined in claim 1, wherein said resorbent material comprises collagen.

3. The surgical dental graft appliance as defined in claim 1, wherein said bone granularized bone augmentation material comprises granules of human bone.

4. The surgical dental graft appliance as defined in claim 1, wherein said granularized bone augmentation material comprises a mixture of bone granules and human blood,

5. The surgical dental graft appliance as defined in claim 1, wherein said granularized bone augmentation material comprises a mixture of human bone granules, artificial bone granules and human blood.

6. The surgical dental graft appliance as defined in claim 5, wherein said resorbent material comprises collagen.

7. The surgical dental graft appliance for augmenting bone as defined in claim 1 wherein said resorbent material forms flexible walls to said vessel.

8. The surgical dental graft appliance for augmenting bone as defined in claim 1, wherein said resorbent material forms walls for said vessel that are non-self-supporting and wherein said vessel expands to a size that fills said open window and pushes into contact with said sinus membrane.

9. The surgical dental graft appliance for augmenting bone as defined in claim 1, wherein said resorbent material defines walls for said vessel that are self-supporting.

10. The surgical dental graft appliance for augmenting bone as defined in claim 9, wherein said vessel comprises a size that fills said open window and pushes into contact with said sinus membrane and said maxillary bone.

11. The surgical dental graft appliance as defined in claim 1, wherein said non-metal vessel includes an extending tab for tacking said appliance to the maxillary bone of the patient.

12. The surgical dental graft appliance as defined in claim 1, wherein said vessel defines a predetermined volume, and further comprises an entrance for admitting a plunger spout to access said predetermined volume and press a fill of bone granules out into said volume.

13. The surgical dental graft appliance as defined in claim 1, wherein said graft appliance is also for closing an open window formed in the wall of a patient's maxilla bone bordering the patient's sinus cavity during the course of a sinus grafting procedure; and wherein said vessel is of a size to fit in and fill said window.

14. A surgical dental graft appliance for promoting bone growth and closing a hole formed during the course of a sinus grafting procedure through the upper ridge of the jawbone and opening in the region of the sinus cavity, irrespective of the physical integrity of the sinus membrane along the side of said sinus cavity, comprising:
an elongate flexible walled sleeve-like vessel, said sleeve-like vessel being of a size to fit in and extend through said hole and into said sinus cavity, said vessel being formed of a resorbent material that is slowly resorbed in body fluids and that is porous to human bone cells and blood cells to permit migration thereof out of and into said sleeve-like vessel; and
granularized bone augmentation material, said granularized bone augmentation material being retained in and filling said sleeve-like vessel, said granularized bone augmentation material growing to the person's skull over a substantial period of time period while said sleeve-like vessel is being resorbed within the body.

15. In the Window procedure for maxillary sinus bone augmentation in which a window is formed into the sinus cavity and a portion of the Schneiderian membrane is released from a portion of the cavity wall to form a ancillary cavity inferior to the sinus cavity that is accessible through said window and thereby reduce the size of the sinus cavity, the step of inserting a bag of bones into said ancillary cavity, said bag being formed of collagen and said bones comprising a mixture of human blood and bone granules.

16. In the osteotome procedure for maxillary sinus bone augmentation in which a passage is formed through the ridge in the upper jaw and exits into a region inferior to the sinus cavity between a lifted portion of the Schneiderian membrane that borders the sinus cavity and the underlying maxilla sinus bone, the step of forming a bag of bones in said passage and extending into said region, said bag being formed of collagen and said bones comprising a mixture of human blood and bone granules.
